(19) United States
(12) Patent Application Publication
(10) Pub. No.: US 2017/0079800 A1
(43) Pub. Date: Mar. 23, 2017

(54) Hybrid Femoral Component Design for a Hip Prosthesis and Method of Stem Insertion

(71) Applicant: Michael Messieh, Clermont, FL (US)

(72) Inventor: Michael Messieh, Clermont, FL (US)

(21) Appl. No.: 14/969,510

(22) Filed: Dec. 15, 2015

Related U.S. Application Data
(60) Provisional application No. 62/221,724, filed on Sep. 22, 2015.
(60) Provisional application No. 62/239,001, filed on Oct. 8, 2015.

(51) Int. Cl. A61F 2/36

(52) U.S. Cl. CPC A61F 2/3672; A61F 2002/3678; A61F 2002/3664

(57) ABSTRACT

An improved hybrid femoral component design for a hip prosthesis has a cross section having a rectangular or other shape lateral end and a circular or elliptical medial end, where the medial anterior-posterior (AP) dimension of the implant is greater than the lateral AP dimension of the implant. The cross sectional axis of the circular or elliptical medial end may be inclined or partially offset in relation to the axis of the rectangular lateral end. The lateral side of the proximal stem may have a plurality of shapes. This hybrid proximal stem design lends itself to a hybrid proximal femoral preparation surgical technique where the medial calcar may be power reamed and the lateral femur and canal may be broached.

### CROSS REFERENCE TO RELATED APPLICATIONS

The present application claims the benefit of the filing date of U.S. Provisional Patent Application Ser. No. 62/221,724, filed Sep. 22, 2015, and U.S. Provisional Patent Application Ser. No. 62/239,001, filed Oct. 8, 2015. The foregoing applications are incorporated by reference in their entirety as if fully set forth herein.

### STATEMENT REGARDING FEDERALLY SPONSORED RESEARCH OR DEVELOPMENT

Not applicable.

### REFERENCE TO A MICROFICHE APPENDIX

Not applicable.

### TECHNICAL FIELD

The present invention relates generally to medical and surgical devices, and more particularly to an improved hybrid femoral component design for a hip prosthesis, and a method for stem insertion.

### BACKGROUND INFORMATION AND DISCUSSION OF RELATED ART

The present invention relates to the femoral component of a hip prosthesis. It is widely known that the success of cementless hip replacement depends on the correct sizing and placement of the prosthesis within the medullary cavity of the femur. Fixation of the stem may rely on the distal fixation or a midstem twist or proximal fixation based on oval, rectangular or wedge shapes. The proximal part of the stem of the femoral component of the prosthesis should provide support and stability. The size and shape of the proximal femur can vary widely. The stem in the proximal region should have a configuration to allow for better fit and stability of the stem within the femoral intramedullary canal with minimal removal of strong bone. The present invention was made in view of drawbacks with prior femoral components attempting to achieve stability.

For example, United States Patent Application No. 2014/0343685 by Ranawat, et al. discloses a femoral component for a hip prosthesis has a proximal end for disposition at a superior position in a femur and a distal end opposite the proximal end. The femoral component has opposite medial and lateral sides and opposite anterior and posterior faces extending between the medial and lateral sides. At least a proximal part of the femoral component has a first superior-to-inferior taper so that a medial to lateral dimension decreases gradually from the proximal end toward the distal end, a second superior-to-inferior taper so that an anterior to posterior dimension of the femoral component decreases gradually from the proximal end toward the distal end and a lateral-to-medial taper so that an anterior to posterior dimension of the femoral component decreases gradually from the lateral side toward the medial side.

U.S. Pat. No. 5,358,534 to Dudasik, et al. describes a femoral component for a hip prosthesis having a distal portion defining a central longitudinal axis. The component has a generally conically shaped mid-shaft portion and a proximal portion with a shape based on the reamer and a shaped chisel used by the surgeon to prepare the proximal metaphysis and medullary canal. The proximal portion is shaped in a manner wherein a cross-section taken perpendicular to the central axis has a medial side formed as a first circular arc, a corner of the cross-section formed by the posterior and lateral sides as a second circular arc with a center on the central axis. The posterior side is arcuate and concave and tangent to the first and second circular arcs, with the anterior side being arcuate and convex.

The foregoing references reflect the current state of the art of which the present inventor is aware. Reference to, and discussion of, these references is intended to aid in discharging Applicant's acknowledged duty of candor in disclosing information that may be relevant to the examination of claims to the present invention. However, it is respectfully submitted that the above-indicated references do not disclose, teach, suggest, show, or otherwise render obvious, either singly or when considered in combination, the invention described and claimed herein.

### SUMMARY OF THE INVENTION

The present invention provides an improved hybrid femoral component design for a hip prosthesis, and a method for stem insertion. The hybrid design cross section has a rectangular or other shape lateral end and a circular or elliptical medial end, where the medial anterior-posterior (AP) dimension of the implant is greater than the lateral AP dimension of the implant. The cross sectional axis of the circular or elliptical medial end may be inclined or partially offset in relation to the axis of the rectangular lateral end. The lateral side of the proximal stem may have a plurality of shapes.

This hybrid proximal stem design lends itself to a hybrid proximal femoral preparation surgical technique where the medial calcar may be power reamed and the lateral femur and canal may be broached. This hybrid surgical technique may have distinct advantages, including but not limited to better implant bone ingrowth, less stem malalignment especially in varus, faster femoral preparation, less risk of femoral fracture, greater stability, and may provide a quick guide to final stem size needed for stability based on the diameter of the medial reamer.

The modification to the stem may be applied to most existing hip stems so each manufacturer does not have to develop a new stem design but just add this modification for greater stability, which is a significant advantage.

Rationale for the Inventive Hip Stem Design

The use of short hip stems is rising because of the increasing popularity of MIS surgical techniques, particularly the direct anterior approach to hip replacement surgery. There are many potential benefits for proximal fixation with a MIS technique. Shortening the stem may avoid distal potting with subsequent loosening, thigh pain and femoral fracture and proximal stress shielding. Short hip stems require stability in the proximal femur which is less predictable than distal fixation.

Proximal fixation with a short stem is challenging because of the variations in proximal femur anatomy, bone quality, surgical technique, and current implant designs. The main stresses on the implant with short stems are axial and rotational stresses in a proximal femur with several unpredictable preop variables.

Subsidence can be addressed by proper sizing, medial stem to bone contact and a tapered design in several dimensions. Posterior shear may be addressed by minimizing the posteromedial implant bone gap. The options include a wide medial stem, a curved stem or posteromedial wedge. As an example, the Accolade 2 design compared to Accolade 1 is shorter, greater medial to lateral dimensions stem proportions. This addresses the need for a better medial gap contact. The stem may be shortened without too much loss in stability.

The inventive design addresses posterior shear secondary to inadequate implant to bone contact. Rotational instability of a hip stem may be decreased with a longer stem with distal fixation, a curved stem, a larger proximal dimension, a large lateral dimension, a large medial dimension or fins.

The fixed posteromedial augment acts as a doorstop. Although this necessitates right and left femurs, this modification of existing designs is advantageous over new hip designs with symmetrically wide medial dimensions. The stem is more intuitive, less costly, bone conserving, familiarity, and optional: if the standard stem is felt to be unstable the augmented design may be used. The augment may be used to adjust for excessive retroversion or anteversion. The right may be used on the left hip and vise versa if the surgeon feels that may achieve the desired version.

It is therefore an object of the present invention to provide a new and improved hybrid femoral component design for a hip prosthesis.

It is a further object of the subject invention to provide a new and improved method for stem insertion.

Other novel features which are characteristic of the invention, as to organization and method of operation, together with further objects and advantages thereof will be better understood from the following description considered in connection with the accompanying drawings, in which preferred embodiments of the invention are illustrated by way of example. It is to be expressly understood, however, that the drawings are for illustration and description only and are not intended as a definition of the limits of the invention. The various features of novelty which characterize the invention are pointed out with particularity in the claims annexed to and forming part of this disclosure. The invention resides not in any one of these features taken alone, but rather in the particular combination of all of its structures for the functions specified.

There has thus been broadly outlined the more important features of the invention in order that the detailed description thereof that follows may be better understood, and in order that the present contribution to the art may be better appreciated. There are, of course, additional features of the invention that will be described hereinafter and which will form additional subject matter of the claims appended hereto. Those skilled in the art will appreciate that the conception upon which this disclosure is based readily may be utilized as a basis for the designing of other structures, methods and systems for carrying out the several purposes of the present invention. It is important, therefore, that the claims be regarded as including such equivalent constructions insofar as they do not depart from the spirit and scope of the present invention.

Further, the purpose of the Abstract is to enable the U.S. Patent and Trademark Office and the public generally, and especially the scientists, engineers and practitioners in the art who are not familiar with patent or legal terms or phraseology, to determine quickly from a cursory inspection the nature and essence of the technical disclosure of the application. The Abstract is neither intended to define the invention of this application, which is measured by the claims, nor is it intended to be limiting as to the scope of the invention in any way.

Certain terminology and derivations thereof may be used in the following description for convenience in reference only, and will not be limiting. For example, words such as “upward,” “downward,” “left,” and “right” would refer to directions in the drawings to which reference is made unless otherwise stated. Similarly, words such as “inward” and “outward” would refer to directions toward and away from, respectively, the geometric center of a device or area and designated parts thereof. References in the singular tense include the plural, and vice versa, unless otherwise noted.

### BRIEF DESCRIPTION OF THE DRAWINGS

The invention will be better understood and objects other than those set forth above will become apparent when consideration is given to the following detailed description thereof. Such description makes reference to the annexed drawings wherein:

FIG. 1 is a view of a modified reverse trapezoidal shape for a hybrid femoral component for a hip prosthesis of this invention;

FIG. 2 is a view of an alternate embodiment illustrating that the medial end may be oval or circular, and may be off axis at any angle; and

FIG. 3 is a view of a reamer device that may be used to prepare the proximal femoral canal.

### DETAILED DESCRIPTION OF THE INVENTION

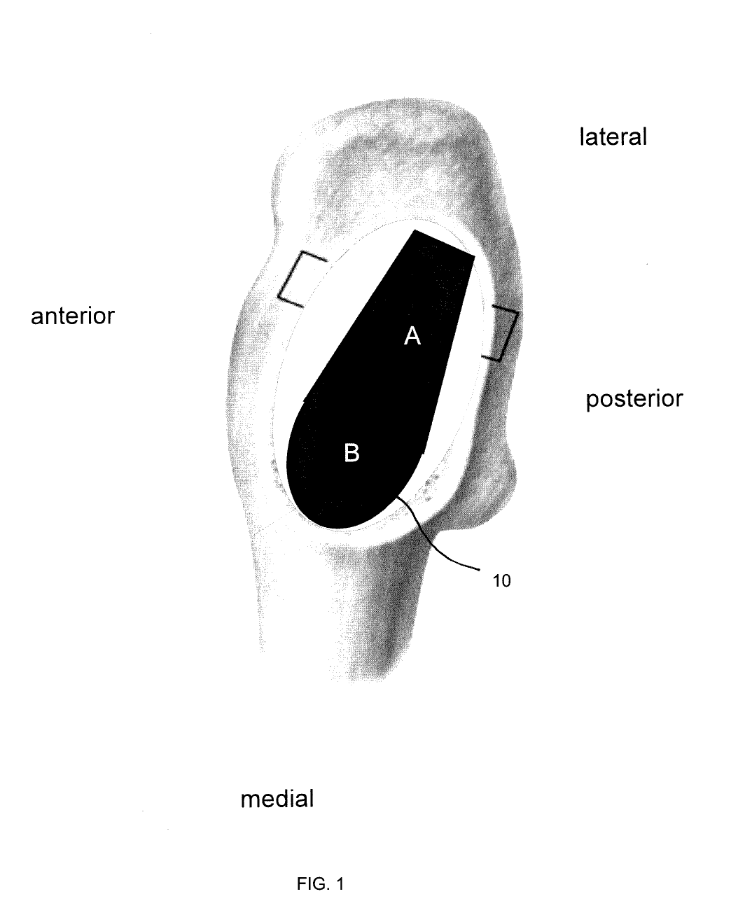

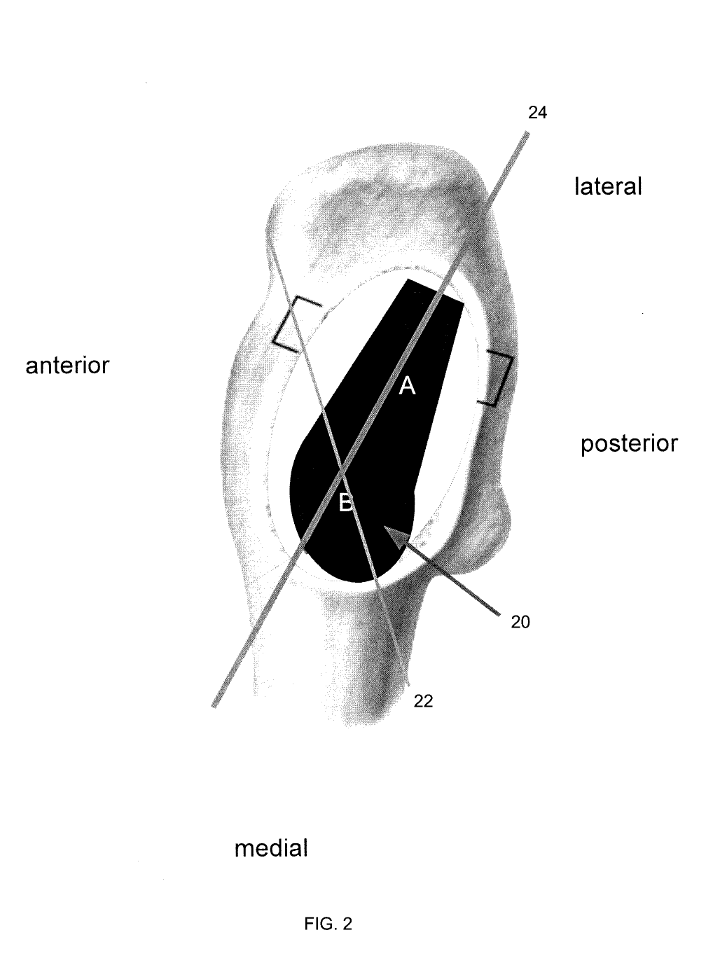

Referring to FIGS. 1 and 2, there is illustrated therein a new and improved hybrid femoral component design for a hip prosthesis. FIG. 1 is a view of a modified reverse trapezoidal shape for a hybrid femoral component 10 of this invention, with a rectangular cross sectional lateral end A and a circular cross sectional medial end B; and FIG. 2 is a view of an alternate embodiment 20 illustrating that the medial end B may be oval in cross section, and may be off axis at any angle.

The hybrid design cross section has a rectangular or other shape lateral end (part A) and a circular or elliptical medial end (part B), where the medial anterior-posterior (AP) dimension of the implant is greater than the lateral AP dimension of the implant. The cross sectional axis 22 of the circular or elliptical medial end (part B) may be inclined or partially offset in relation to the cross sectional axis 24 of the rectangular lateral end (part A). The lateral side of the proximal stem may have a plurality of shapes.

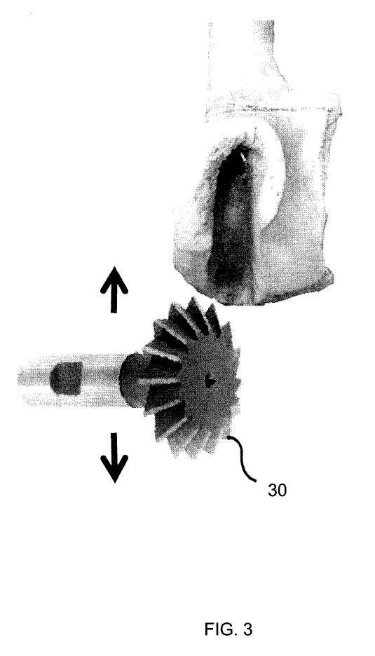

FIG. 3 is a view of a reamer device 30 that may be used to prepare the proximal femoral canal. This hybrid proximal stem design lends itself to a hybrid proximal femoral preparation surgical technique where the medial calcar may be power reamed and the lateral femur and canal may be broached. This hybrid surgical technique may have distinct advantages, including but not limited to better implant bone ingrowth, less stem malalignment especially in varus, faster femoral preparation, less risk of femoral fracture, greater stability, and may provide a quick guide to final stem size needed for stability based on the diameter of the medial reamer.

The advantages of the inventive hybrid design include, but are not limited to:

The greater stability of the proximal design will allow shorter length femoral stems to be used. Short length stems are popular when hip replacement surgery is performed through the anterior surgical approach.

A rectangular or trapezoidal cross section is expected to have high implant stress levels. The circular or elliptical medial shape is expected to decrease the stress. This design is expected to have a low displacement when loaded and low stress values because of the larger AP dimension medially compared to laterally and the circular or elliptical shape medially.

The simplicity of the geometry decreases stress concentrations.

The hybrid design has a relatively greater cross sectional area compared to rectangular designs which are commonly used which is more favorable.

Less femoral bone is removed from the lateral end compared to the medial end proximally. This has distinct advantages: less soft tissue dissection is needed, there is less risk of a femoral bone fracture, and there is less risk of a greater trochanteric bone fracture. Instrumentation to remove bone is relatively difficult to use on the lateral side compared to the medial side of the femur. With the popularity of the anterior hip surgical approach gaining, femoral implants that rely on more bone removal from the lateral side have a distinct disadvantage.

When hip replacement surgery is performed through the anterior surgical approach, the femoral canal with most femoral implants is prepared with various sized and shaped hand broaches placed on curved handles. This technique is imprecise and may lead to varus implant positioning, femoral fracture, and undersizing of the implant, and may pose a challenge to accessing the femoral canal in certain patient conditions such as obesity and muscular patients. In addition, proximal and distal femoral geometry mismatch is a challenge that may need distal canal reaming for some implants. Using a short stem implant with improved proximal stability based on this hybrid design may allow the surgeon to avoid having to ream the distal canal.

The circular or elliptical medial end (part B) may be partially offset in relation to the lateral rectangular end to allow the use of left or right femoral stem implants.

This hybrid design can be applied to many existing commonly used short stem implant designs tailored for the anterior hip approach. The modification of adding a circular or elliptical medial end (part B) inline or partially offset in relation to the axis of the lateral rectangular end to existing implants is a novel idea, with a distinct advantage. For example, in some cases, the implant manufacturer offers femoral implants with a standard or large proximal body. If the implant is not stable after surgical insertion, the surgeon may insert a larger implant. This may result in a femoral fracture or a poorly seated stem that results in a leg length inequality. With this hybrid design, the surgeon may chose the same sized implant with a circular or elliptical medial end inline or partially offset in relation to the axis of the lateral rectangular end to increase implant stability without having to use a larger or longer stem. More patients with differing proximal femoral geometries may be successfully accommodated with this approach compared to the difficulty of storing different implant femoral stem designs in the operating room or using a larger implant with possible complications as described above.

The circular or elliptical medial end (part B) may be inline or partially offset in relation to the axis of the lateral rectangular end (part A) which lends itself to a distinct advantage when preparing the proximal femoral canal. The medial end of the proximal femur (often referred to as the calcar area) is easier to access with power reamers compared to the lateral side when hip replacement surgery is performed through the popular anterior surgical approach. Reaming the medial calcar area for femoral stem preparation has been described and proven to be reliable and stable. An example of this is the instrumentation used in the SROM system from DePuy. The hybrid design has a distinct advantage since the medial end is circular/elliptical which lends itself to power reaming which is safer and more precise than rasping. In addition, the stem size can be generally determined by using various diameters of reamers medially since the lateral end AP dimension is narrower. Since the medial end is oversized compared to the lateral end, sizing is primarily determined by the medial end dimensions. Power reaming of the proximal femur has distinct advantages but is not commonly used through the anterior surgical approach because the torso/pelvis are often in the way. The medial calcar area is often easily accessible to power reaming The hybrid proximal stem design lends itself to a hybrid femoral preparation technique where the medial calcar may be power reamed and the lateral femur and canal may be broached. This hybrid surgical technique may have distinct advantages: better bone ingrowth, less stem malalignment especially in varus, faster femoral preparation, less risk of femoral fracture, and may provide a quick guide to final stem size needed for stability based on the diameter of the medial reamer.

The inventive design may be used as a modification to most existing designs so a completely newly designed stem may not be necessary. This will help in several ways: 1) help with FDA clearance based on predicate stem designs; 2) manufacturers may decide to apply this modification to just a select number of stem sizes or stem designs to limit cost of extra inventory or address the needs/preferences of select surgeons who want to use this design without the company having to make a significant capital investment; and 3) add this modification to existing implants that the surgeon is already familiar with but is seeking this extra feature so that more patients can qualify for the stem design without having to use a completely different stem design to attain implant stability. Companies can add this modification to any of their stems, decide whether it is effective, strategic or economical in their particular case and with minimal impact on manufacturing process.

Some prior designs may lock-in femoral stem anteversion in a position that may be different than the desired one. To compensate for this potential problem, the neck of the stem may need to be placed not inline with the midline of the medial stem. This results in the inventive design where the medial circle/oval can be placed midline to the stem or offset to it, so that anteversion may be better adjusted.

The inventive design removes less proximal bone, and applies only to the proximal femoral stem while prior medial designs are along the entire length of the stem.

Prior designs have the potential for windshield wiper effect of the lateral stem. The negative implications of this are not yet known but may be associated with femoral fracture, stem subsidence or loosening. In addition, prior designs may be more difficult to insert in hard sclerotic bone and narrow proximal femurs.

Very importantly, the surgeon may use a standard femoral stem trial initially of his choice. If this trial is unstable with rotational stress, the surgeon may choose to use the stem trial with the inventive modification and recheck stability. This is a logical graduation process. But if the surgeon uses prior designs, the surgeon must remove a large amount of bone medially initially which may not be necessary or desirable.

The inventive reamer design for stem insertion may be used for the inventive design or prior designs. The reamer wheel can rotate forward, backward or oscillate back and forth and tipped at any angle to create a more precise cavity for the stem for improved stability and avoid need for straight reamers that are often possible to use with the anterior approach. The width and diameter of the wheel are related to the stem implant to aid with sizing.

The inventive design is like adding a doorstop to the proximal side of a hip stem. The doorstop concept is key since posterior shear on the stem may result in loosening. Since the hip forces are mostly posterior and medial, a complete medial circle like prior designs are not always needed since a doorstop is usually placed on the side of the door to resist the expected force, i.e., to resist the door from moving in a particular direction not both directions.

Reverse Proximal Hip Design and Method of Insertion.

The importance of proximal stem design to resist hip loading forces: Femoral hip stem implant stability at the time of surgery is critical to the success of total hip replacement. Short hip stems are becoming popular with minimally invasive total hip replacement surgery. The effect of stem shortening on initial stability may affect biomechanical performance. Micromotion of the stem is expected to increase as the stern length is shortened, A short stem should achieve physiological conditions in the proximal femur after implantation without compromising primary stability. The initial stability of cementless hip stems requires resistance to axial and torsional loading. Torsional loading has been shown to cause the largest displacements at the bone-implant interface. Therefore, large torsional loads, e.g. stair climbing, must be avoided initially and stem design must optimize stability. The geometry of the proximal stem significantly affects the primary stability and can be important in the prevention of excessive micromotion. Short stems that provide a more proximal fit and fill because of an expanded proximal design concept are favorable for rotational stability, If cortical contact is achieved with press-fit, stability may not be compromised. Interference fit in proximally fixed hip stem reduces the effects of gap on stability. The prosthesis-bone inducible micromotion under load causes a gap opening in the medial region, and shear slippage in the posterior region. Design optimization addressing these gap areas may be beneficial. Resistance against irreversible migration into retroversion of short stems is necessary to avoid subsidence, dislocation and femoral bone fracture.

The inventive design adds a medial and/or postermedial circular or elliptical tapered build up to the proximal hip stem to provide medial/posteromedial cortical contact for greater implant stability, less stem bone gaps, less medial bone stress shielding and more physiologic medial bone loading, thereby improving the stability of existing short stern and quasi-stable implants. This may be a better strategy than simply using larger stem implant sizes to achieve stability that may result in incomplete stem seating, femoral fracture, leg length inequality, or necessity to ream the distal canal.

The importance of proximal stem design to maintain femoral stem anteversion: Universal short stems (not specifically made for right or left hips) may have a tendency to be placed in neutral, excessive anteversion or retroversion (−10 to +20 degrees) depending on patient, surgeon and instrumentation factors. Excessive anteversion may result in implant loosening by increasing torque when the hip is loaded and excessive retroversion may result in a hip dislocation. The inventive design adds a medial and/or postermedial circular or elliptical tapered build up to the proximal hip stem that may resist stem retroversion and the anterior posterior width such as to restore acceptable anteversion.

The importance of surgical technique: In addition to optimal stem design, implant material and bone quality, surgical technique should be precise. Interfacial gaps between the hip stem and femoral bone may occur with the conventional broach/rasp systems when preparing the femoral cavity. A poor broaching technique may result in large gap formations, stem undersizing, malalignment, femoral perforation, femoral fracture and errors in stem version. If done properly, there is a high correlation between torsional stability of the rasp/broach and stability of the corresponding hip stem. Broaching the canal consistently well depends on a number of factors including surgeon skill, instrumentation, bone quality, femoral proximal distal mismatch, patient size and weight, hip stiffness and distance between the proximal femur and pelvis.

The complications of broaching may be effectively reduced by the inventive hybrid method of reaming the medial proximal femur and broaching a narrower lateral proximal femoral. cavity. This is considered a novel hybrid technique which may improve primary stability by achieving medial and posteromedial cortical contact. Rigid fixation of the implant may result with the proposed line-to-line reaming technique of the medial posterior medial proximal femur. The medial posterior medial proximal AP width of the implant may be related to remainder of the implant as a ratio. This has a definite advantage. Reaming the medial posterior medial proximal femur area with a straight or tapered reamer will give the surgeon an estimate of the final implant size early in the femoral preparation stage which is markedly advantageous. Of course, the surgeon may elect to use broaches which are shaped to match the proximal stem design as well.

Summary of benefits of the inventive design and technique:

Hypertrophy or lack of resorption of the medial proximal femoral cortex: As the joint load is shifted to the medial cortex, thickening of the medial cortex is expected, which could diminish stress-shielding of the proximal femur.

The inventive design may be considered as an internal collar without the disadvantages of an external collared stem.

An eccentric circular elliptical medial proximal stem has numerous advantages, including but not limited to:

a. increased stability thru medial and posterior cortical contact; it is tapered for axial loading and eccentric posterior to resist posterior shear forces;

b. the medial proximal femur is reamed for more precise bone contact compared to rasping/broaches;

c. the narrower lateral width of the proximal stem preserves bone laterally by being wider medially;

d. the wider medial/posterior width is expected to promote medial bone hypertrophy and less stress shielding. This design promotes neutral or slight valgus alignment of the stem which may be beneficial;

e. the stable short stem design address the problems with proximal distal mismatch and avoids need for distal canal reaming;

f. the implant design builds in acceptable anteversion and prevents retroversion and resists retroversion often seen in straight stems;

g. the level of press-fitting during stem insertion is critical and is optimized by this design which is better than using a universally increasing stem size to achieve stability;

h. the width of the medial stem may reflect the size of the final stem inserted which provides the surgeon with information about stem size early in the procedure.

The above disclosure is sufficient to enable one of ordinary skill in the art to practice the invention, and provides the best mode of practicing the invention presently contemplated by the inventor. While there is provided herein a full and complete disclosure of the preferred embodiments of this invention, it is not desired to limit the invention to the exact construction, dimensional relationships, and operation shown and described. Various modifications, alternative constructions, changes and equivalents will readily occur to those skilled in the art and may be employed, as suitable, without departing from the true spirit and scope of the invention. Such changes might involve alternative materials, components, structural arrangements, sizes, shapes, forms, functions, operational features or the like.

Therefore, the above description and illustrations should not be construed as limiting the scope of the invention, which is defined by the appended claims.

What is claimed as invention is:

1. A femoral component design for a hip prosthesis comprising:
a femoral component having a lateral end and a medial end, the medial end having a circular or elliptical cross section, where the medial anterior-posterior dimension is greater than the lateral anterior-posterior dimension.

2. The femoral component design for a hip prosthesis of claim 1 wherein the lateral end has a rectangular cross section.

3. The femoral component design for a hip prosthesis of claim 1 wherein the cross sectional axis of the medial end is offset in relation to the cross sectional axis of the lateral end.

4. A method for stem insertion for a femoral component for a hip prosthesis comprising:
a hybrid proximal femoral preparation surgical technique where the medial calcar is power reamed and the lateral femur and canal may be broached.
